(19) United States
(12) Patent Application Publication
(10) Pub. No.: US 2025/0127607 A1
(43) Pub. Date: Apr. 24, 2025

(54) TISSUE REPAIR SYSTEM

(71) Applicant: TENDOMEND LTD., Nazareth (IL)

(72) Inventors: Shahar PELED, Beit Hashitta (IL); Maor Itzhak ROSENBERG, Haifa (IL); Ilan GRUNBERG, Haifa (IL); Yuval Zekharya SHEZIFI, Haifa (IL)

(73) Assignee: TENDOMEND LTD., Nazareth (IL)

(21) Appl. No.: 18/695,825

(22) PCT Filed: Sep. 27, 2021
(86) PCT No.: PCT/IB2021/058780

§ 371 Date: Mar. 27, 2024

(30) Foreign Application Priority Data
Sep. 30, 2020 (GB) 2015530.5

(51) Int. Cl. A61F 2/08; A61B 17/04

(52) U.S. Cl. CPC A61F 2/0811; A61B 17/04; A61B 2017/0464; A61F 2002/0823; A61F 2002/0852; A61F 2002/0864

(57) ABSTRACT

A system for tissue repair includes a tissue fixation device having two adjustable self-locking loops, a tensioning device having one fixed loop and one adjustable self-locking loop and a connector having one fixed loop.

### BACKGROUND

The present invention relates to a tissue repair system and methods of using same. Embodiments of the present invention relate to a tissue repair system that includes a tissue fixation device a tensioning device and a connector and to methods of utilizing the system to repair ruptured/severed tissues such as tendons.

Tendon injuries are common and are associated with pain, reduced mobility, and reduced use of the affected body parts.

Tendon injuries oftentimes require tedious and difficult surgical procedures to repair especially in the case of flexor tendons. Flexor tendons are located within a fibrous tendon sheath that forms a smooth, tight tunnel around the tendon. The tendon sheath ensures that the tendon is in the proper place during movement. The sheath also includes a plurality of discrete fibrous segments referred to as pulleys that translate tendon pull into joint motion. Pulleys provide a mechanical advantage by maintaining the tendons close to the joint's axis of motion while preventing bow-stringing of the tendon away from the bones.

In order to repair a ruptured tendon, a surgeon must find both ends of the tendon, pull the ends through the tight tendon sheath and pulleys and connect the two ends using sutures or anchors. Repair can result in a bulky and/or rough repair site that can abut the edges of the pulleys and limit glide through the tendon sheath. Abutment of the repair site against the pulley can also cause damage or irritation to the repair site during flex resulting in fibrosis and adhesions that oftentimes necessitate additional surgery to correct.

While reducing the present invention to practice, the present inventors have devised a soft tissue repair approach that can be used to repair tendons without the aforementioned limitations of currently used approaches.

### SUMMARY

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIGS. 1A-3 illustrate construction of each of the components of one embodiment of the present tissue repair system.

FIGS. 4A-T illustrate repair of a severed tendon using one embodiment of the present tissue repair system.

FIGS. 5A-N illustrate repair of a severed tendon using another embodiment of the present tissue repair system.

FIGS. 6A-7B illustrate embodiments of a jig that can be used for guiding manual delivery of the present tissue repair system into tissue.

FIG. 7 illustrates ex-vivo repair of a tendon using the present approach.

FIG. 8 is a prior art image illustrating a braid having a bifurcated loop.

### DETAILED DESCRIPTION

The present invention is of a tissue fixation system which can be used to repair soft tissue. Specifically, the present invention can be used to reconnect ruptured or severed tendons such as flexor tendons.

The principles and operation of the present invention may be better understood with reference to the drawings and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

The goals of flexor tendon repair are to promote intrinsic tendon healing and minimize extrinsic scarring in order to optimize tendon gliding and range of motion. Over the past decades numerous repair approaches have been developed and used in pursuit of the ultimate repair approach. The “grasping technique” described by Isidor Kessler and Fuad Nissim in 1969 is a popular method of flexor tendon repair and has been modified over the years to what is at present considered the benchmark—the ‘Modified Kessler Technique’.

In the Modified Kessler Technique a suture is passed through the cut tendon end and out the upper surface of the tendon. The suture is then passed transversely across the tendon to create a grasping loop. The free end is then passed into the tendon and out at the cut end. The suture is then used to repeat the procedure for the other segment of the cut tendon. This procedure is repeated four times to generate a “Four Strands Core Suture”. Then, the free ends of the suture are pulled to approximate the tendon segments. The suture ends are then tied to secure the approximated tendon segments.

While the modified Kessler Technique and its variants provide a somewhat mechanically stable connection between severed tendon segments, they require skill and are time consuming to perform correctly.

While reducing the present invention to practice the present inventors devised a soft tissue repair approach that traverses the above limitations of prior art approaches while providing the following advantages:
(i) fixation of soft tissue such as severed tendon segments without altering tendon profile and limiting glide through the tendon sheath;
(ii) consistent and accurate tendon fixation without having to rely on individual surgical skills;
(iii) system components are soft and non-tissue traumatic;
(iv) unique threading and looping of the fixation system through the soft tissue ensures mechanical stability and provides superior anchoring that facilitates early active rehabilitation; and
(v) can be used in a minimally invasive approach that is less traumatic to the surgical site and patient.

Thus, according to one aspect of the present invention there is provided a tissue repair system that can be used in repair of severed or torn tissues. The present repair system is highly suited for use in soft tissue such as tendons such as hand Zone II flexors but can also be used in repair of hand extensors, foot tendons other tendons and ligaments.

The present tissue repair system relies strand fixation of each of the two severed tendon ends around two axis. Each of the severed tendon ends is secured by a strand loop or loops and the strand loops are fixed in position by one or more additional strands that are positioned through the tendon and around (and perpendicular to) the strand loops. The severed tendon ends are then attached to each other via strand ends that are routed through the tendon and out of the tendon ends.

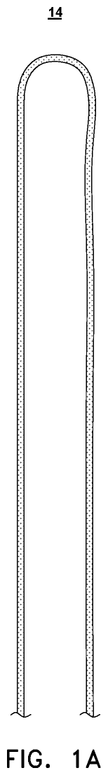

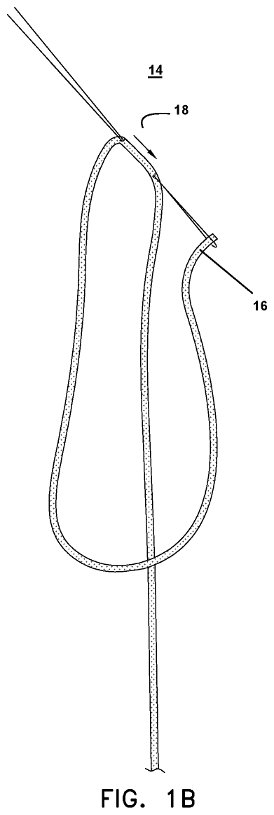

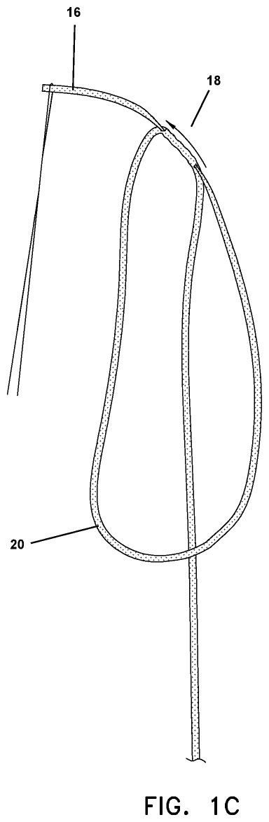

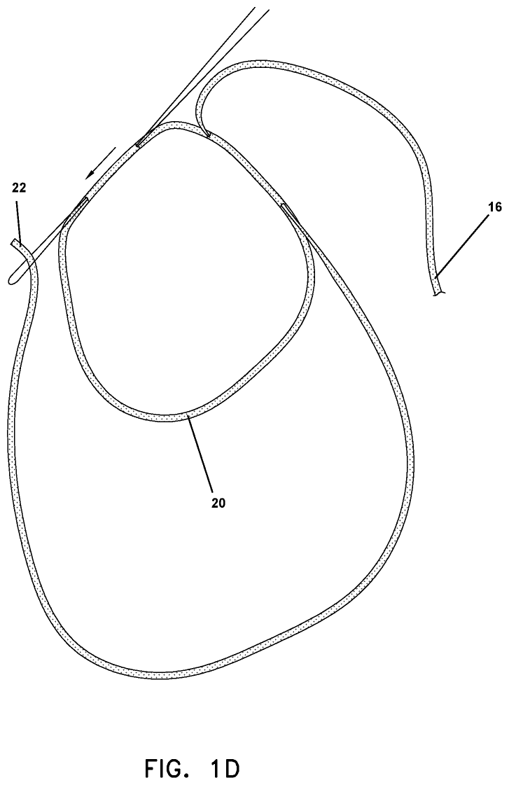

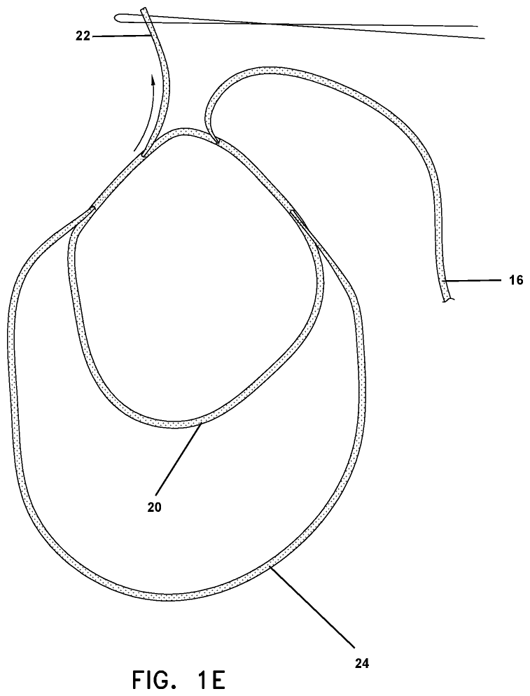

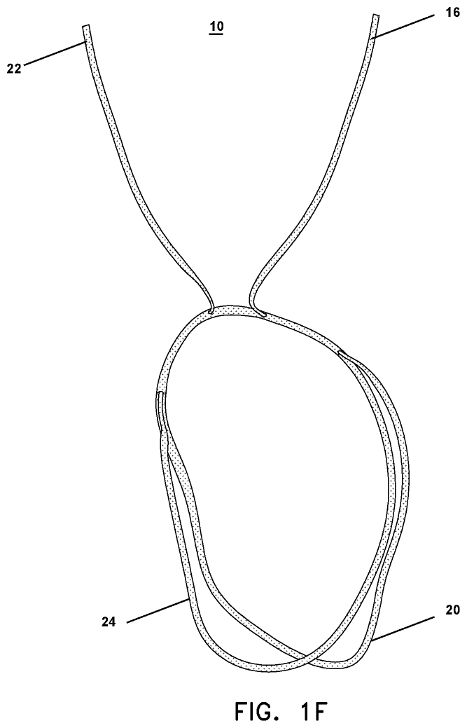

According to one embodiment of the present invention, the tissue repair system of the present invention includes a tissue fixation device having two adjustable loops (adjustable self-locking loops, e.g., whoopie slings), a connector that includes a single fixed loop and a tensioning device having a single adjustable loop and a single fixed loop (e.g., Brummel loop). The configuration and construction of each of these system components is described hereinbelow with reference to FIGS. 1A-3.

These three components of the present tissue repair system function collectively as a tissue anchor. By using two tissue repair systems each anchored to, for example, an end of a severed tendon, the present invention can be used to interconnect severed tendon ends in a manner that is mechanically robust and does not alter tendon profile or limit glide through the tendon sheath.

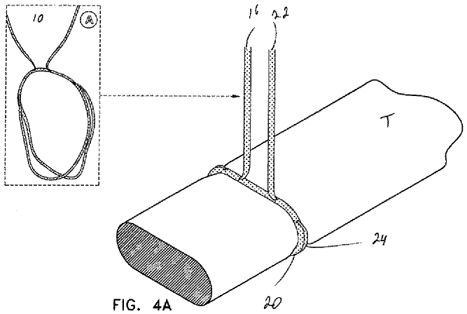

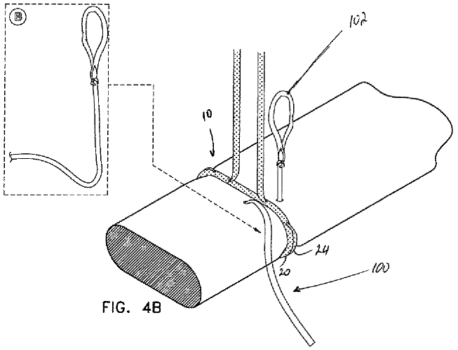

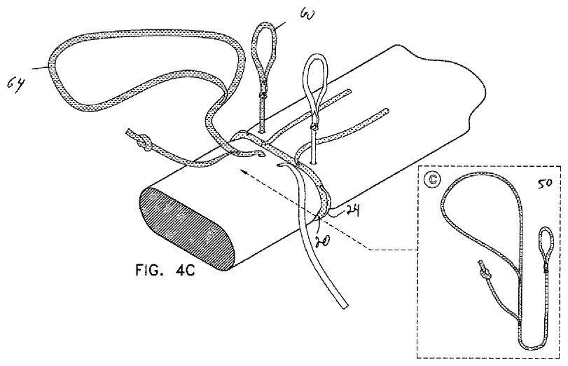

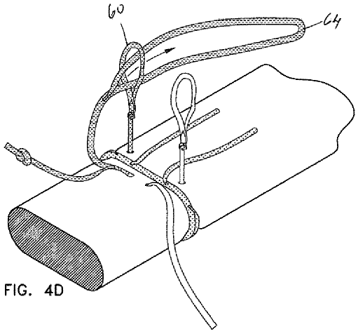

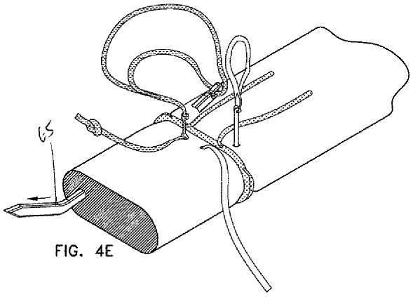

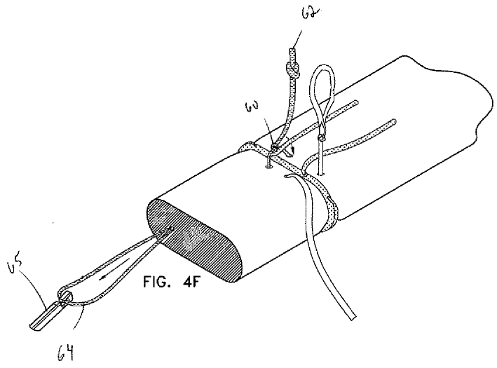

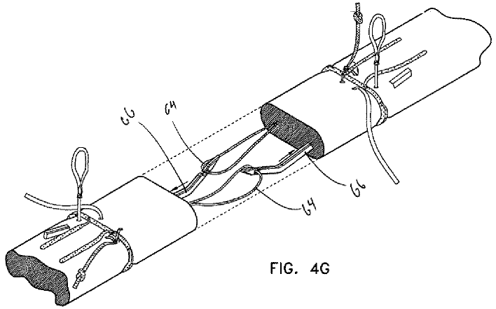

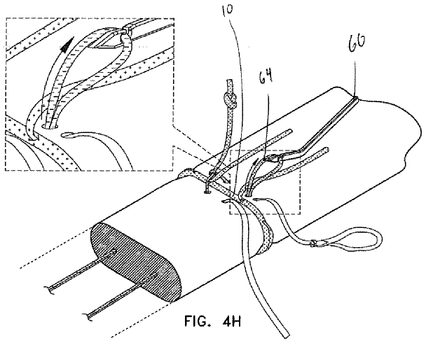

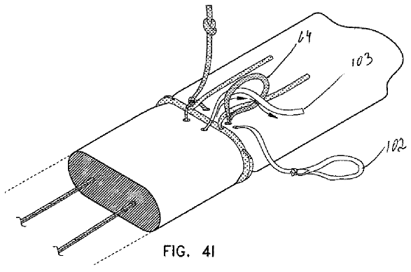

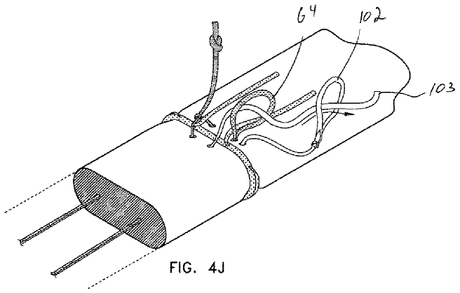

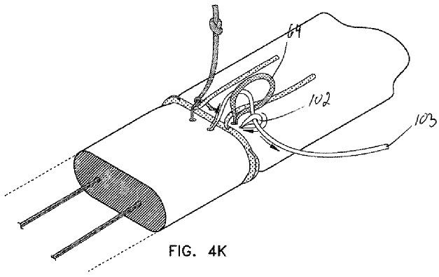

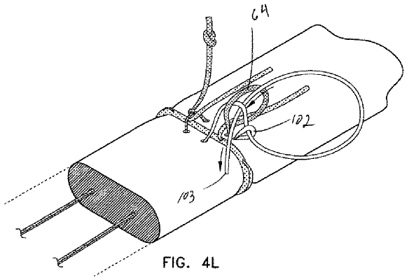

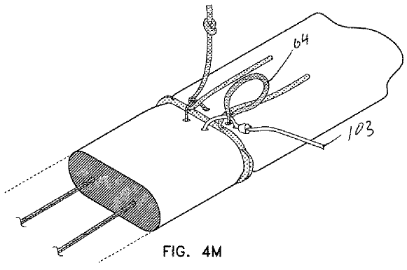

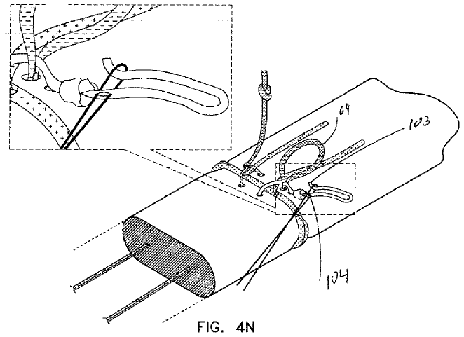

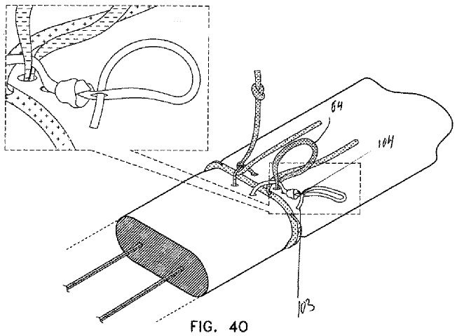

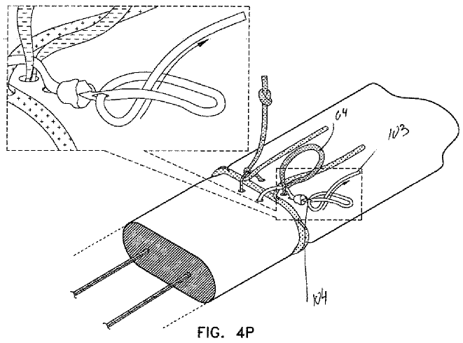

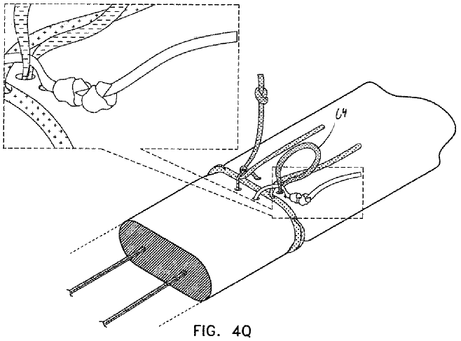

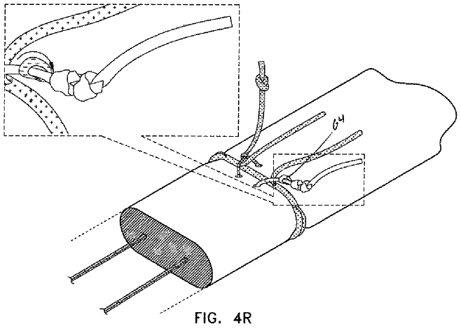

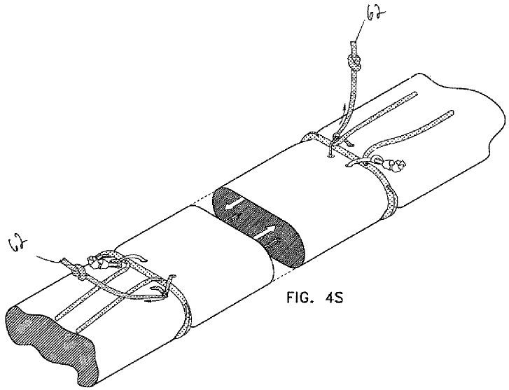

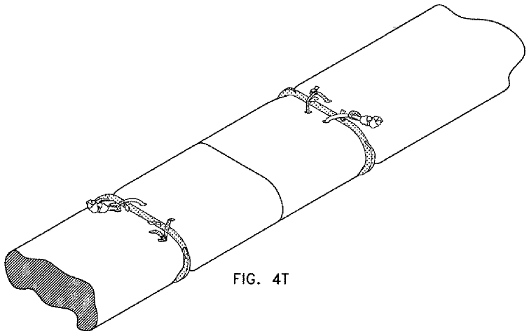

The two adjustable self-locking loops of the tissue fixation device are positioned around the tissue, the connector is threaded into and out of the tissue on both sides of the tissue fixation device on one side of the tendon portion and the tensioning device is similarly threaded into and out of the tissue on both sides of the tissue fixation on an opposite side of the tendon portion. The adjustable loops of the tissue fixation device apply a constricting force to the tissue while the connector and tensioning device maintain the adjustable loops of the tissue fixation device in position (e.g., prevent the loops from sliding along the tissue). The adjustable self-locking loop of the tensioning device is then threaded through the tendon and out of the severed tendon end. This process is repeated with a second system on the second tendon portion and the two adjustable self-locking loops of the tensioning device are then threaded through the opposite tendon ends and out of the tendon tops. The system can then be tensioned to approximate the tendon ends and tied off to secure the newly formed connection. A detailed description of this process is provided hereinbelow with reference to FIGS. 4A-T.

By using these three components to both secure the circumference of the tendon and provide through tissue anchoring, the present system provides a secure connection without substantially altering the profile of the tissue.

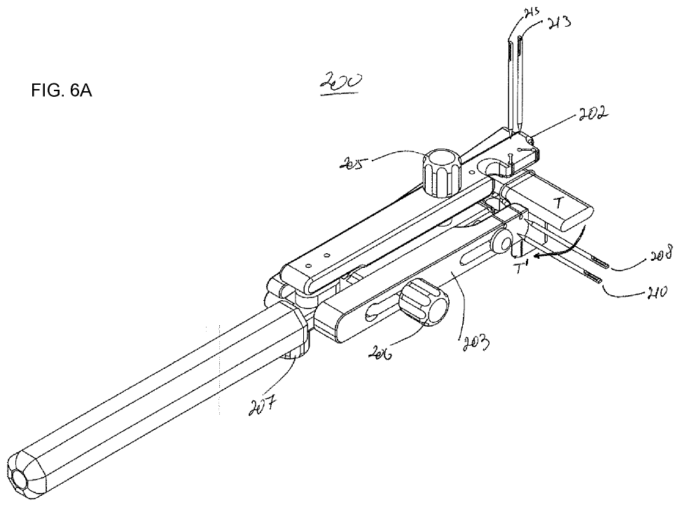

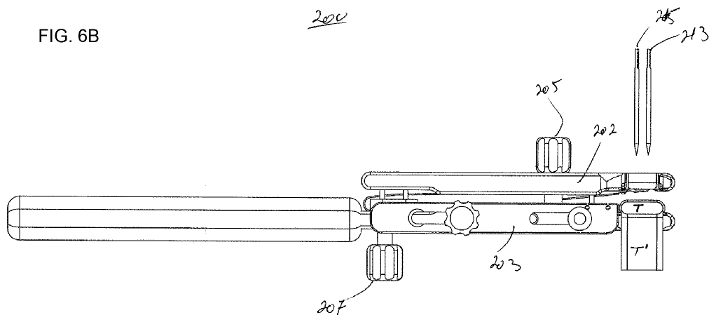

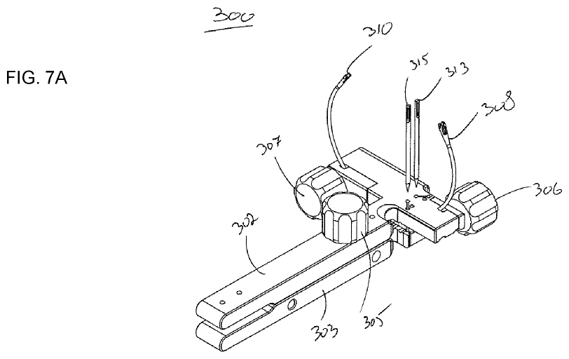

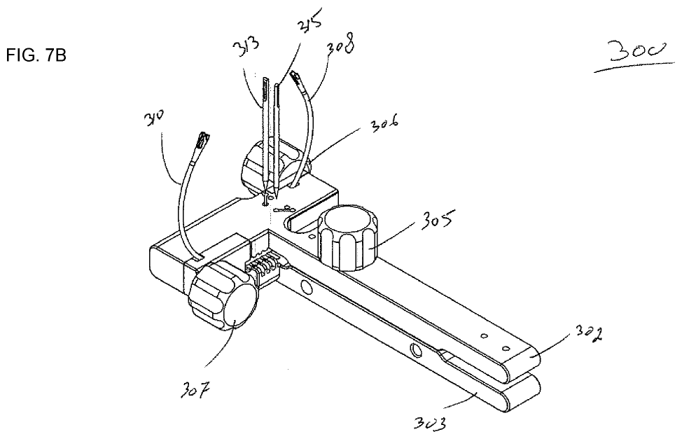

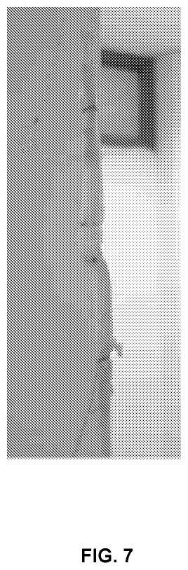

The present tissue repair system can be anchored to soft tissue such as tendon manually or via a guide jig or by using a combination of manual and guided delivery. An exemplary jig is described in detail hereinbelow with reference to FIGS. 6A-7B.

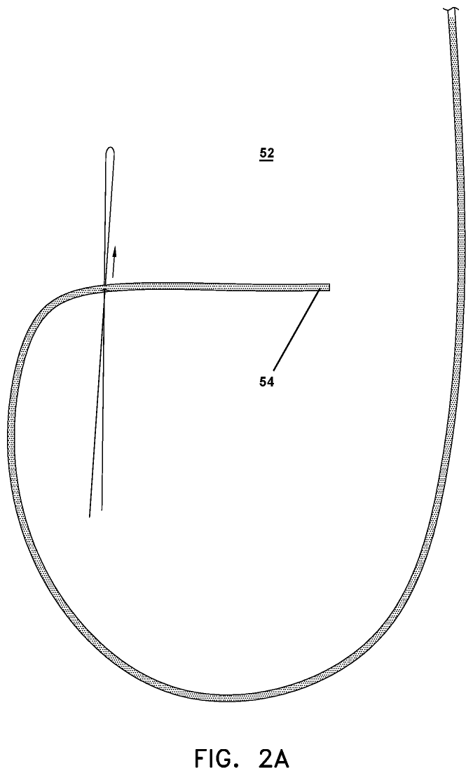

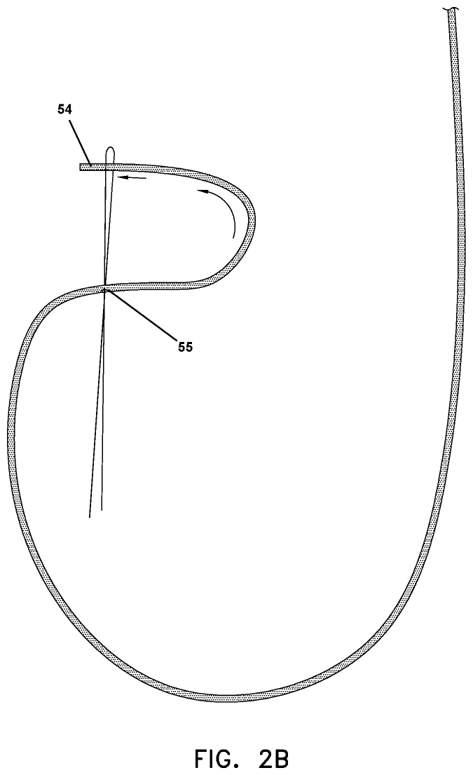

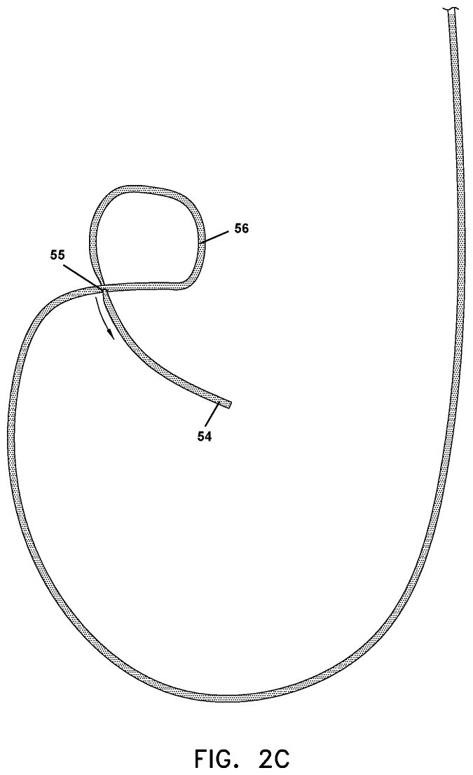

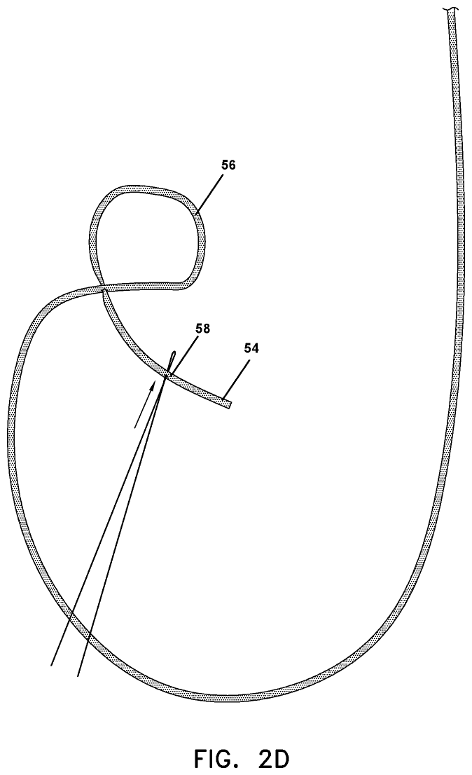

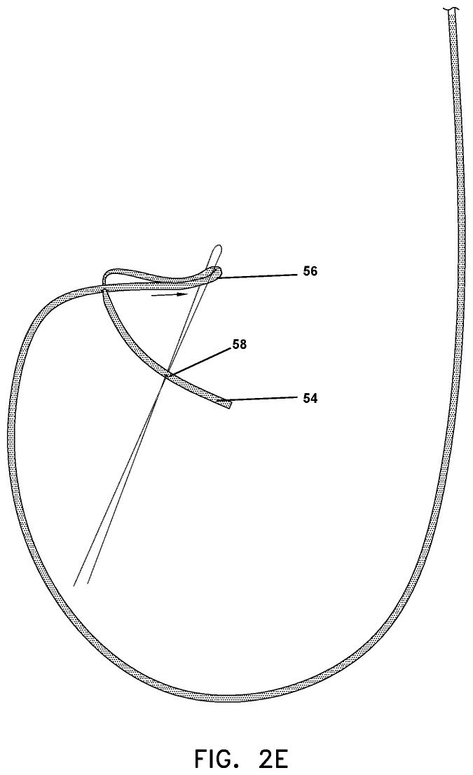

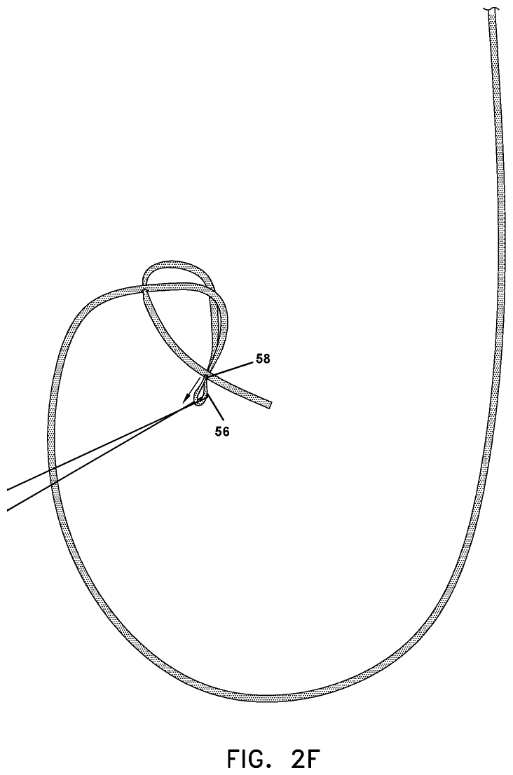

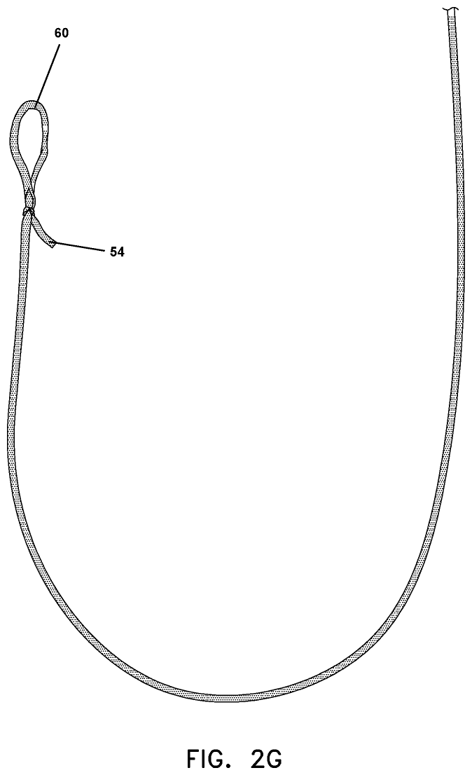

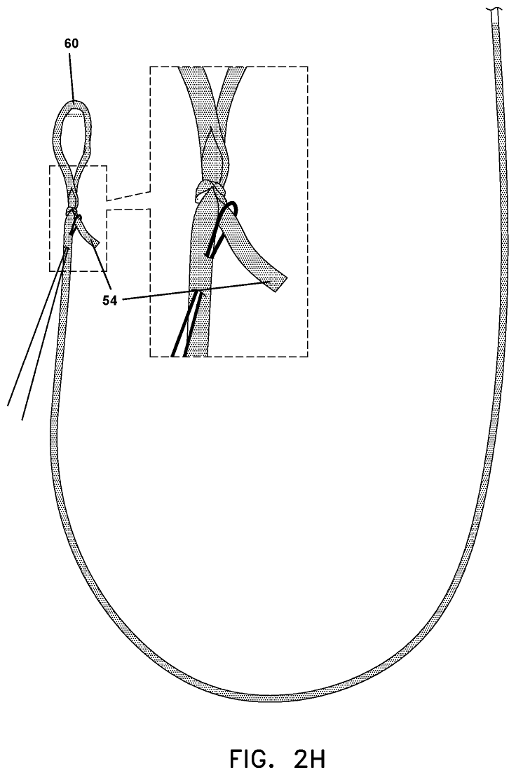

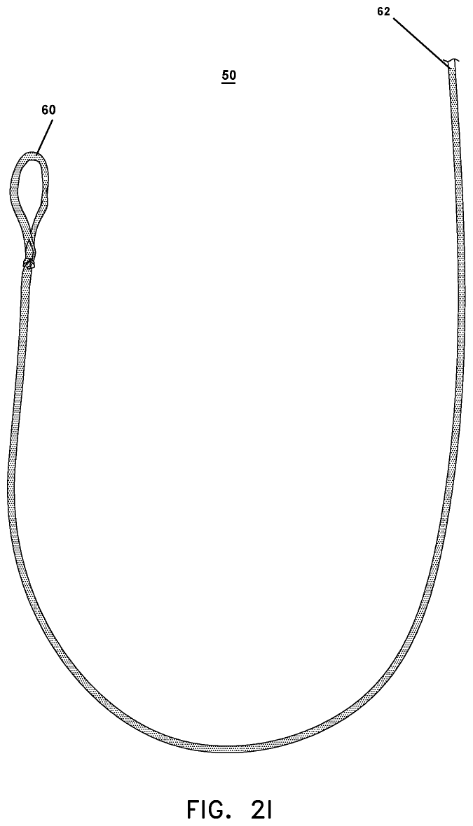

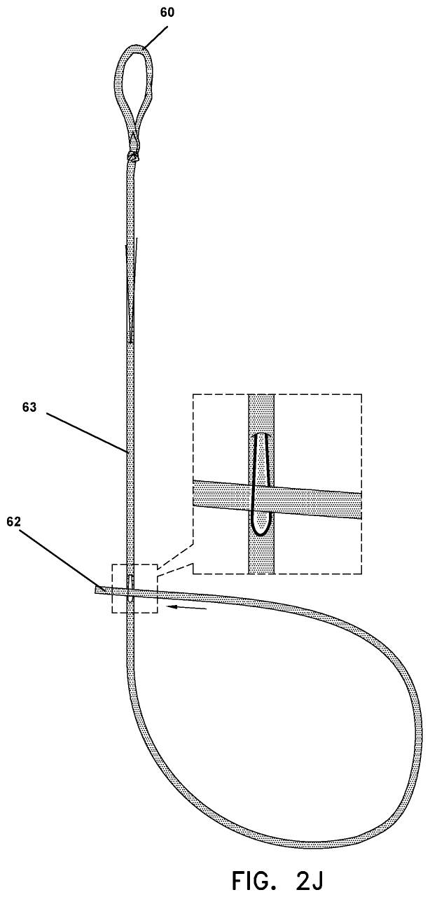

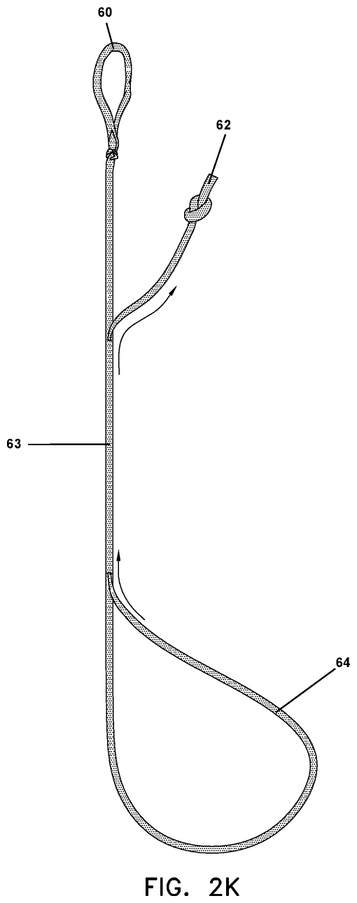

Referring now to the drawings, FIGS. 1A-3 illustrate construction of the tissue fixation device (FIG. 1F), the connector (3) and the tensioning device (FIG. 2K) of one embodiment of the present tissue repair system.

FIGS. 1A-F illustrate construction of the tissue fixation device which is referred to herein as device 10. Device 10 is constructed from a single non-rigid and non/semi-elastic cord 14 (FIG. 1A) that is fabricated from, for example, polyester, polyethylene, polypropylene, Silk, UHMWPE and the like. Cord 14 can be fabricated from 8-16 braided filaments forming a hollow Herringbone pattern at, for example, 55-80 Peaks Per Inch (PPI). Cord 14 can also be formed from twisted or twined filaments. Cord 14 can be 150-200 mm in length and 0.15-0.4 mm in diameter with a USP Knot Break between 1 to 4 (lbs.).

As is shown in FIG. 1B, a first end 16 of cord 14 is spliced through a mid-portion 18 of cord 14 to create a first adjustable self-locking loop 20 (FIG. 1C). Second end 22 of cord 14 is then similarly spliced through cord 14 (FIG. 1D) to create a second adjustable self-locking loop 24 (FIG. 1E). Each of loops 20 and 24 is separately adjustable to a self-locking diameter. Loops 20 and 24 can be positioned and locked around a tendon portion as is further described hereinbelow with reference to FIG. 4A. Loops 20 and 24 can be Whoopie slings, ladder-lock buckle ratchet tie or a zip tie.

FIGS. 2A-K illustrate construction of a tensioning device which is referred to herein as tensioning device 50 or tensioner 50.

Tensioning device 50 is manufactured from a cord 52 (FIG. 2A) which is identical to cord 14 in material properties (construction and tensile strength) and design (e.g., hollow braid) but is 150-200 mm in length.

As is shown in FIGS. 2A-B, a first end 54 of cord 52 is threaded through a hole 55 in cord 52 and a formed loop 56 (FIG. 2C) is threaded through a hole 58 at end portion of cord 52 (FIGS. 2D-F) to form a fixed loop 60 (FIG. 2G). First end 54 is then spliced through cord 52 as is shown in FIG. 2H to furnish the fixed loop of tensioning device 50 (FIG. 2I).

The second free end 62 is spliced through a mid-portion 63 of cord 52 to create an adjustable self-locking loop 64 (FIG. 2K).

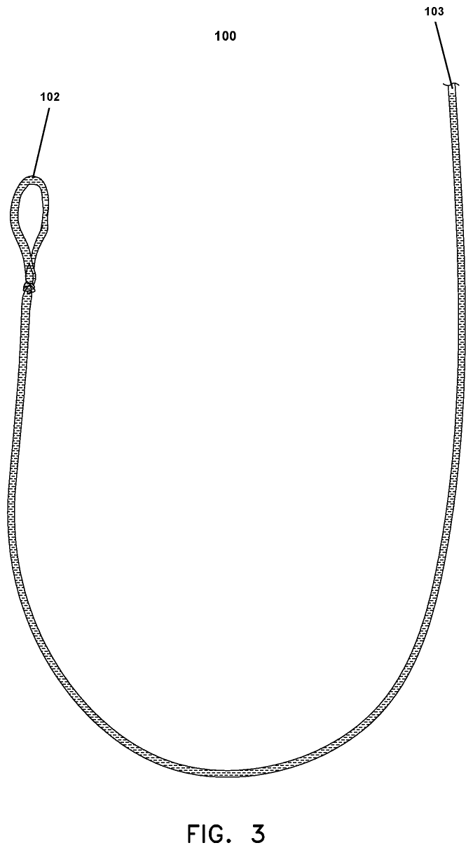

FIG. 3 illustrates construction of a connector device which is referred to herein as connector 100 or device 100.

Device 100 is manufactured the same as described for device 50 (FIGS. 2A-I).

The self-locking adjustable loops of devices 10 and 50 (20, 24 and 64 respectively) can be adjusted through a diameter range of 0.1-100 mm and can withstand a static load of 4-10 kg, depend on the length of the buried area. Each of fixed loops 60 of tensioner 50 and 102 of device 100 can be 1-5 mm in diameter and can withstand a static load of 8-12 kg, depend on the braid configuration i.e. PPI, # of strands, strands diameter . . . .

As is mentioned hereinabove, the present system is designed for interconnecting severed ends of a tissue such as a tendon. FIGS. 4A-T illustrate the process of positioning devices 10 and 100 and tensioner 50 in and around a severed tendon end and the process of interconnecting two ends of a severed tendon using two units of the present system.

FIG. 4A illustrates anchoring of device 10 to a severed end of a tendon (T). Adjustable self-locking loops 20 and 24 of device 10 are positioned around the stump of the tendon and are tightened while maintaining the relatively oval profile (cross-section) of the tendon.

FIG. 4B illustrates anchoring of device 100 to the tendon. Using a needle (not shown), end 102 of connector 100 is threaded into and out of the tendon around loops 20 and 24.

FIG. 4C illustrates anchoring of tensioner 50 to the tendon. Using a needle (not shown), fixed loop 60 is threaded into and out of the Tendon around loops 20 and 24.

In FIGS. 4D-F, adjustable self-locking loop 64 of tensioner 50 is threaded inside fixed loop 60 and then into the tendon and out of the severed end using needle 65.

Following performing the above steps on the second severed end of the tendon, two needles 66 are inserted into and along the tendon to grip adjustable self-locking loop 64 of tensioner 50 and to pull it inside and over device 10 (FIGS. 4G-H).

Free end 103 of device 100 is inserted through adjustable self-locking loop 64 of tensioner 50 and through fixed loop 102 of device 100 as is shown in FIGS. 4I-J. Excess thread of connector 100 is collected and fixed loop 102 is pooled towards the tendon surface (FIG. 4K).

Connector 100 is then secured by performing two adjustable overhand knots. The first knot is a regular knot (FIGS. 4L-M) while the second knot is performed by threading free end 103 through hole 104 (FIGS. 4N-P). FIGS. 4Q-R illustrate tightening of adjustable self-locking loop 64 of tensioner 50. The same procedure is repeated on the other tendon stump and the fixation system is approximated by pulling free ends 62 of tensioner 50 as shown is FIG. 4S. When the desired approximation is achieved the free ends are cut and the tendon is now repaired as is shown in FIG. 4T.

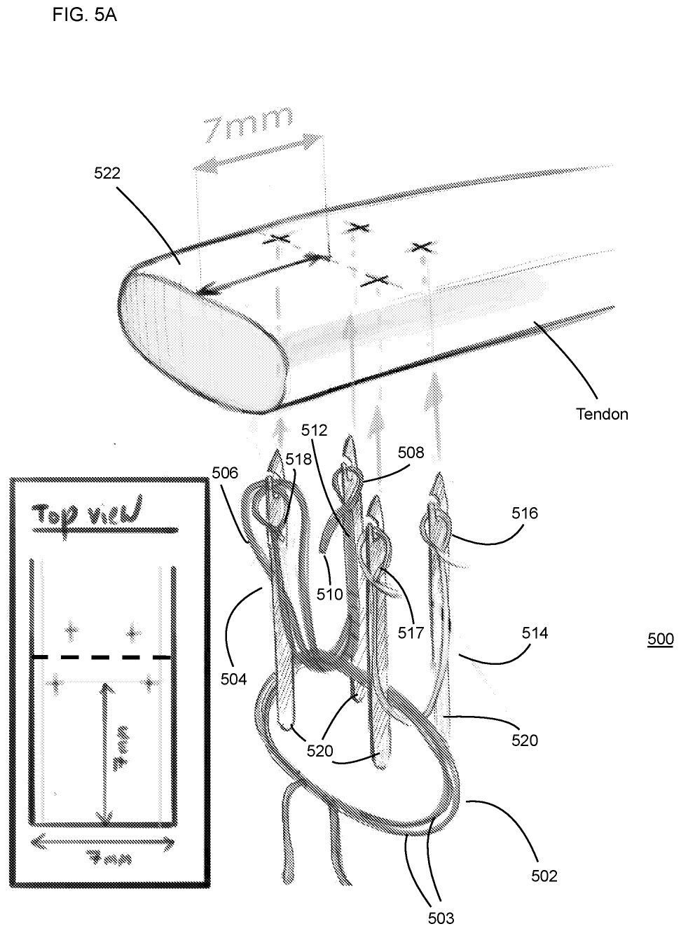

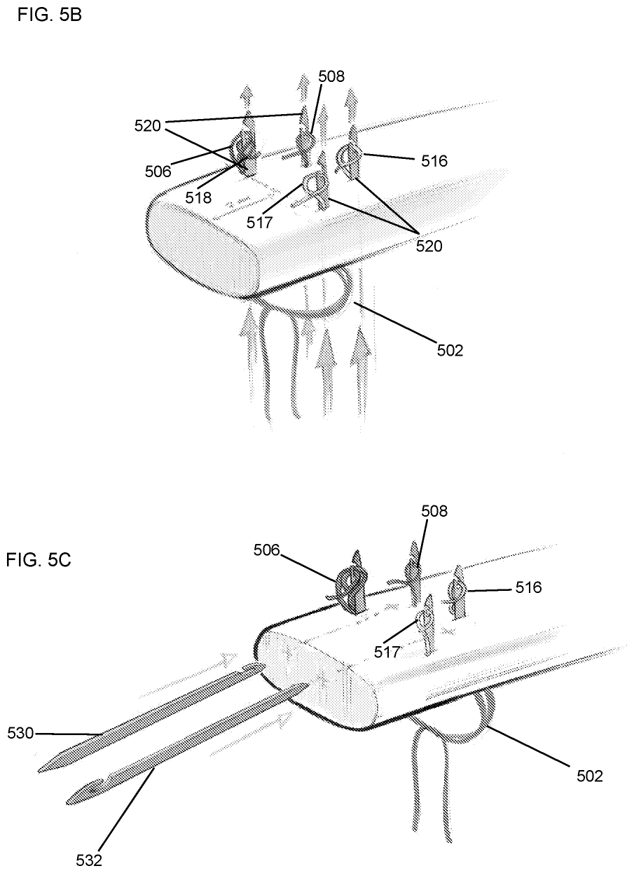

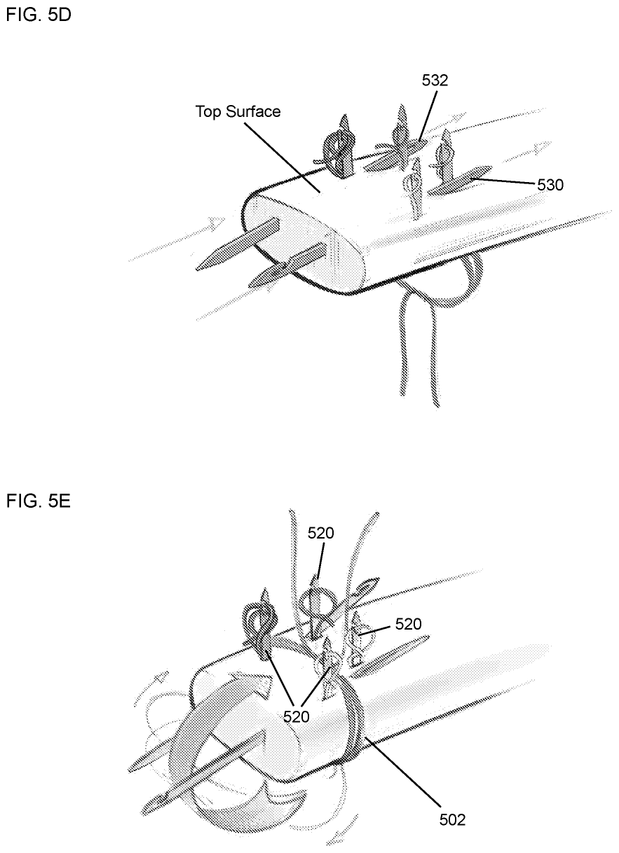

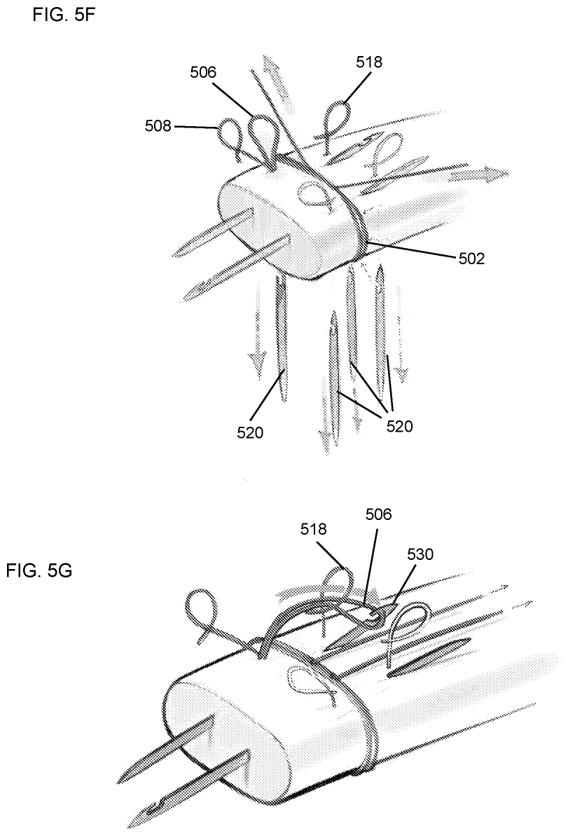

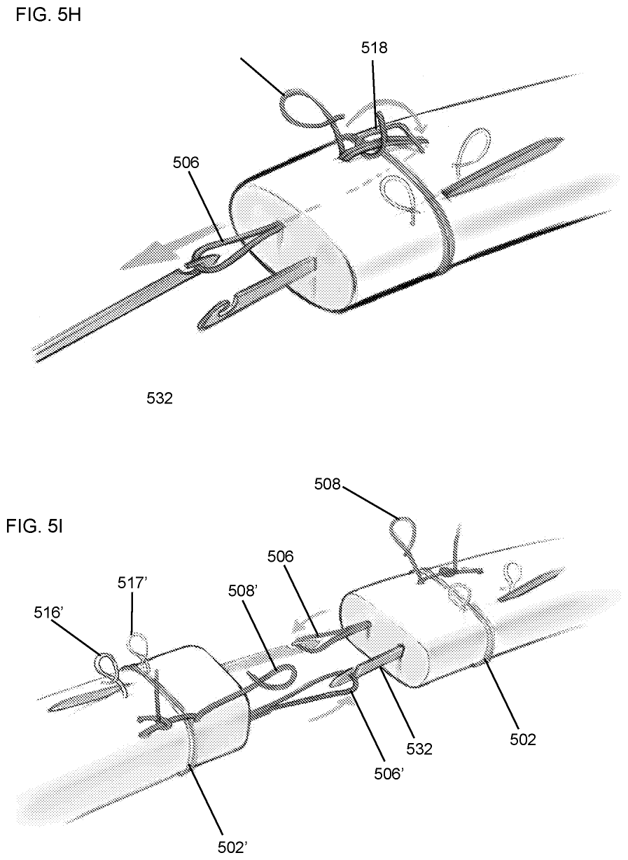

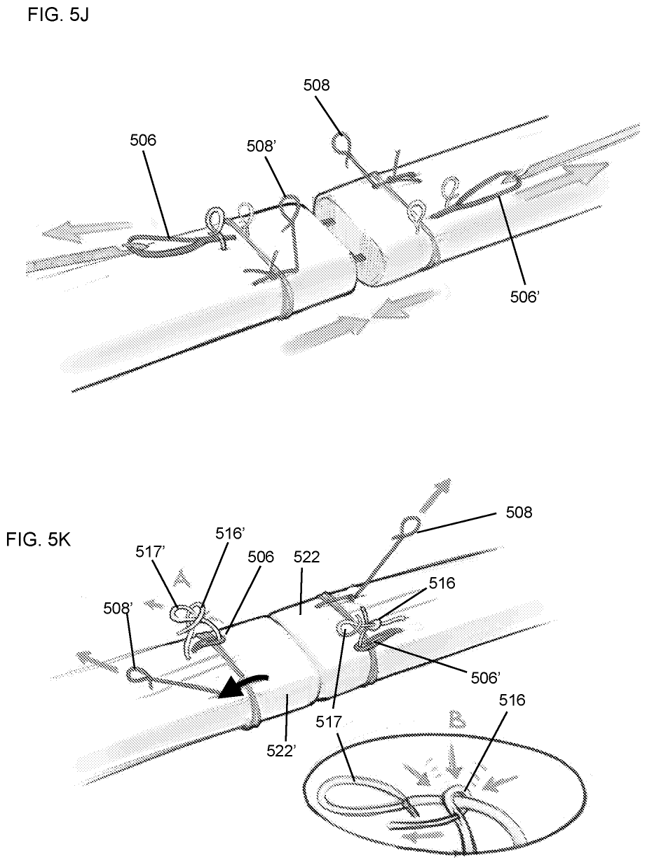

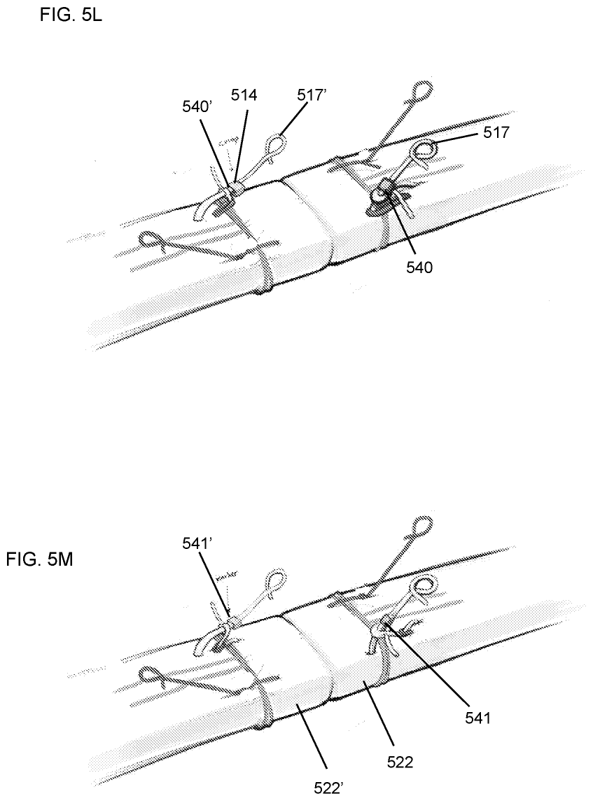

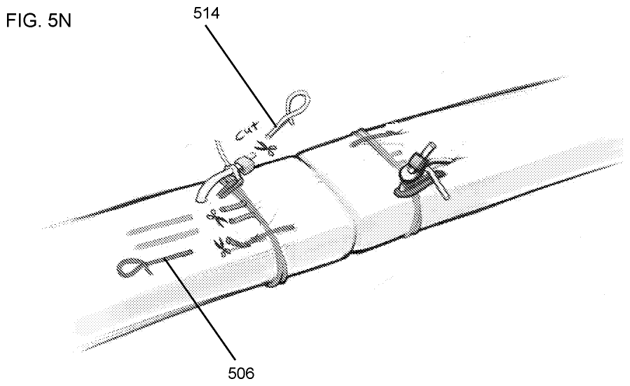

FIGS. 5A-N illustrate another embodiment of the present tissue fixation system and a method of utilizing same for connecting severed tendon ends.

The components of the system are shown in FIG. 5A and include a tissue fixation device 502 including two adjustable self-locking loops 503 (which is similar in configuration to device 10 described above), a tensioning device 504 that includes an adjustable self-locking loop 506 (e.g., whoopie sling), a locking/choking loop 508 that can be fabricated by splicing an end 510 through strand 512, and a connector 514 that includes at least one fixed loop and/or at least one adjustable/choking loop (fixed loop 516 and choking loop 517 are shown). Tensioning device 504 can further include an optional fixed loop 518.

System 500 is shown carried on crochet-type needles 520 that are utilized to position system 500 on a severed end 522 of a tendon. The left bottom panel illustrates the locations through which tensioning device and connector are positioned (via needles 520) through severed end 522 of the tendon. The dotted line shows where tissue fixation device 502 is positioned around severed end 522 of the tendon. The components of system 500 are also deployed on an opposite severed tendon end and are marked 502′, 504′ and 514′.

FIG. 5B illustrates the first step of tendon fixation. Needles 520 are used to push loops 516 and 517 of connector 514 and adjustable self-locking loop 506 (and fixed loop 518) and locking/choking loop 508 of tensioning device 504 through end 522 of the tendon. Connector 514 and tensioning device 504 cradle tissue fixation device 502 and fix it against the lower surface of the tendon.

Needles 530 and 532 (crochet) are forced through the tendon end (FIG. 5C) and out of the top surface of the tendon (FIG. 5D) next to needles 520. Tissue fixation device 502 is flipped around the tendon (FIG. 5E, arrow) and between needles 520.

Needles 520 are removed (FIG. 5F) and adjustable self-locking loop 506 of tensioning device 504 is threaded through locking/choking loop 508 and attached to needle 530 (FIG. 5G). Needle 530 is pulled back to pull adjustable self-locking loop 506 of tensioning device 504 out of end face 532 of the tendon (FIG. 5H).

As is shown in FIG. 5I, the above steps are repeated to a second tendon end 534 with needle 532 used to pull self-locking loop 506′ of tensioning device 504′ into end 522 of the tendon.

Self-locking loops 506 and 506′ are then pulled through respective ends 522 and 534 of the tendon (FIG. 5J). Self-locking loops 506 and 506′ are then connected to connectors 514 and 514′ (respectively) by threading the self-locking loop over a fixed loop of the connector and then threading the fixed loop (516) of the connector through the choking loop (517) of the connector (FIG. 5K). Such a connection configuration secures the self-locking loop to the connector when the choking loop is locked (Shown in FIG. 5K, bubble inset). Alternative configurations of connector 514 that include adjustable self-locking loops (e.g., Whoopie slings) can also be used for such purposes.

Fixed loops 518 and 518′ are then pulled to rotate (arrow) tensioning devices 504 and 504′ within end 522 and 534 and around tissue fixation devices 502 and 502′ (respectively) and reposition strands 519 and 519′ of tensioning device 504 and 504′ (transition shown between FIGS. 5J and 5K).

Crimping 540, 540′ of connector 514′ (and 514 respectively) can be used to further secure the connection (FIG. 5L). Alternatively, the strands of system 500 components can be knotted. Crimps 540 and 540′ can include radiopaque markers 541 and 541′ (FIG. 5M) for visualization of the tendon post repair. The markers can provide an indication of a gap formed between ends 522 and 522′ over time.

The free ends of the tissue fixation devices, tensioning devices and connectors are cut (FIG. 5N) for both sides and the regions of connection between the tensioning devices and connectors (with crimps 540 and 540′) are rotated into the tendon (not shown).

The approach described above can also be performed using a tensioning device 506 and tissue fixation device 502 on one end of the severed tendon and a connector 514 and tissue fixation device 502 on a second end of the tendon. In such an approach, tensioning device 506 is positioned at a center line (of tendon width) and around a first tissue fixation device 502 affixed to a first severed tendon end while connector 514 is positioned at a center line (of tendon width) and around a first tissue fixation device 502 affixed to a second severed end. Tensioning device 506 and connector 514 can then be attached as described above to approximate and join the tendon ends using a single centerline connection.

Although the approaches described with respect to FIGS. 4A-R and FIGS. 5A-O are slightly different, it will be appreciated that some or all of the components (tissue fixation devices, tensioning devices and connectors) can be interchanged between the two fixation approaches.

The above described tendon attachment approaches can be performed manually with or without a guide jig.

FIGS. 6A-B and FIGS. 7A-B illustrate two embodiments of a guide jig which are referred to herein as jig 200 and 300 (respectively). Jigs 200 and 300 can be used to guide needles when threading various components of the present system through the tissue (e.g., tendon stump).

Jig 200 is illustrated in FIGS. 6A-B. Jaws 202 and 203 are used to fix the tendon (T) in a designated position by tightening screws 205-7. After placing device 10 over the tendon, needle 213 is used to thread fixed loop 102 of connector 100 through the tendon. Another needle (not shown) is used to thread fixed loop 102 back to the upper side of the tendon. Needle 215 is used to thread fixed loop 60 of tensioner 50 through the tendon. Another needle (not shown) is used to thread fixed loop 60 back to the upper side of the tendon. Needles 208 and 210 are used to thread adjustable self-locking loop 64 of tensioner 50 through and along the tendon stumps.

Jig 300 is illustrated in FIGS. 7A-B. This embodiment of the jig enables use of curved needles. Two jaws 302 and 303 are used to fix the tendon in a designated position by tightening screws 305-7. After placing device 10 over the tendon, needle 313 is used to thread fixed loop 102 of connector 100 through the tendon. Another needle (not shown) is used to thread fixed loop 102 back to the upper side of the tendon. Needle 315 is used to thread fixed loop 60 of tensioner 50 through the tendon. Another needle (not shown) is used to thread fixed loop 60 back to the upper side of the tendon. Needles 308 and 310 are used to thread adjustable self-locking loops 64 of tensioner 50 through and along the tendon stumps.

As used herein the term “about” refers to +10%.

Additional objects, advantages, and novel features of the present invention will become apparent to one ordinarily skilled in the art upon examination of the following examples, which are not intended to be limiting.

### EXAMPLES

Reference is now made to the following examples, which together with the above descriptions, illustrate the invention in a non-limiting fashion.

### Example 1

### Tendon Repair Integrity

Experiments were conducted in order to validate the performance of the present tissue repair system. The various components of the present system as well as the complete system were tested for resistance to static load. Tendons from human cadavers and from turkey feet were severed and repaired using the approach described with reference to FIGS. 4A-T. The repaired tendon (FIG. 7) was tested for gap formation under a cyclic load (2000 cycles, 1 KG in 0.2 Hz) mimicking a 6-week rehabilitation period, followed by static loading (over 6 KG). No gap formation of more than 2 mm was observed.

### Example 2

### Construction of the Tissue Repair System

A bifurcated loop braid (cortlandbiomedical.com/braided-biomedical-textiles/) having several loops can be used to construct system components that are threaded through during the procedure. The bifurcated loops enable threading through of the braid without having to stab the braid with a needle (and potentially weaken the braid).

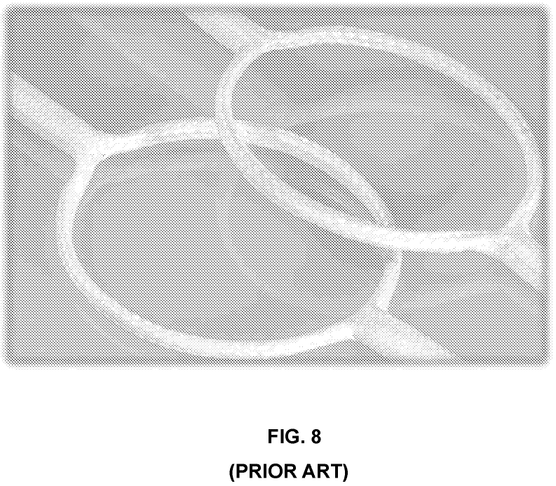

Each loop is generated using the bifurcated loop approach in which two strands are separately braided and repeatedly joined (into a single braid) and split to form the loops (see FIG. 8).

The bifurcated loops are formed at set distances along the braid. The braid containing the loops can then be cut as set distances and each cut portion can then be used to form a single unit. For example, fixed loop 60 or fixed loop 102 can be created from a strand with two loops, 5 mm apart from each-other. These loops can be used as an alternative to holes 55 and 58 stabbed through the braid (FIGS. 2C-D, respectively), an additional loop can be used as an alternative to hole 104 (FIG. 4N).

### Example 3

### Turkey Study

A study was conducted in order to evaluate whether the physical pressure applied by a prototype of the present system on a flexor tendon of turkeys that were subjected to surgically-induced partial lacerations hindered the flow of synovial fluid.

### Materials and Methods

Foot tendons of n=4 male Hybrid turkeys were lacerated and treated in this study, one leg served as a control group and the other as a treatment group. Lacerations in the control group tendons were treated with the Kessler-suture technique while the treatment group was treated using the present system.

### Results and Conclusions

The pressure applied to tendon tissue by the present system did not have any apparent adverse effect on the nourishment of the tendon supplied by the synovial fluid. No progressive inflammatory reaction or necrosis was observed in either of the treatment groups and superficial mature fibrosis was noted in the tendon, at the site of implantation. This data suggests that the external pressure did not induce adverse effects on the tendon and its surrounding tissues.

### Example 4

### Cadaver Tendon Repair

A biomechanical study was performed in order to compare repair strength, finger range of motion and gap formation of a prototype of the present system and a traditional suturing of flexor tendons.

### Materials and Methods

Twelve fresh frozen fingers where used for this feasibility study. Nine tendons were allocated to the present system and three tendons to the traditional 4-strand Kessler and peripheral suture repair. Deep flexors and extensor tendons of the tested fingers were identified and isolated just proximal to the carpal tunnel to allow independent motion of each finger. The repair was performed in zone 2 of the flexor tendon just distal to the A2 pulley but proximal to the insertion of the FDS. The hand was mounted on a flexor simulator and each flexor was tied to an actuator producing 2 cm of excursion. The extensor was connected to a 1 kg weight to extend the finger while the flexor off load. Each finger was cycled for 2000 cycles of flexion-extension simulating 6 weeks of active motion. Following the motion simulator, combined finger range of motion was measured and compared to the pre-operative range. Next, gap formation was measured with an electronic caliper. The tendon was then harvested and a 1 kg load was applied and gap formation was measured. Lastly, we measured the load to failure with an electronic force gauge.

### Results and Conclusions

Combined Finger Range of Motion (ROM) decreased from 234.67±6.51° to 211.67±10.50° for the traditional suture and from 244.0±9.9° to 234.5±5.8° for the present system. While distal A2 pulley venting due to repair buckling was needed for all traditional repair, none of the tendons repaired using the present system needed pulley venting due to the low profile of the repair. Repair using the present system demonstrated gap formation of 0.93±0.18 mm in 3 of 8 specimens after applying 1 kg load. Gap formation was a dynamic phenomenon in all specimens and the gap closed after load removal. Load to failure averaged 7.8±2.36 kg for the present system and 6.76±4.10 kg for traditional repair. Repair failure occurred at the suture material for the present system and at the knot level for the traditional repair.

In conclusion, repair using the present system was found to be superior to traditional repair in that it did not impair finger range of motion even without pulley venting; it demonstrated a dynamic recoiling phenomena wherein the clinically non-significant gap between the stumps dynamically closed after load reduction; it demonstrated higher load to failure with failure occurring in the suture material and not at the knot thus making the repair less reliant on surgeon skill and more reliant on suture material.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

What is claimed is:

1. A system for tissue repair comprising a tissue fixation device including two adjustable self-locking loops and at least one of:
(a) a tensioning device including an adjustable self-locking loop and a choking loop; and
(b) a connector having at least one fixed loop or at least one adjustable self-locking loop.

2. The system of claim 1, wherein each of said tissue fixation device, said tensioning device and said connector is fabricated from a braided material.

3. The system of claim 1, wherein said adjustable self-locking loop is a spliced loop.

4. The system of claim 3, wherein said spliced loop forms a whoopie sling.

5. The system of claim 1, wherein said fixed loop is a Brummel loop.

6. The system of claim 1, wherein said tissue fixation device includes two free ends.

7. The system of claim 1, wherein said connector includes a free end.

8. The system of claim 2, wherein said braided material forms a hollow braid.

9. The system of claim 1, comprising said tissue fixation device, said tensioning device and said connector.

10. A method of repairing a severed tendon comprising:
(a) positioning a first tissue fixation device having two adjustable self-locking loops around a first segment of the severed tendon;
(b) looping a tensioning device through said tendon and around said two adjustable self-locking loops of said first tissue fixation device;
(c) positioning a second tissue fixation device having two adjustable self-locking loops around a second segment of the severed tendon;
(d) looping a connector through said tendon and around said two adjustable self-locking loops of said second tissue fixation device; and
(e) interconnecting said first segment and said second segment of the severed tendon via said tensioning device and said connector.

11. The method of claim 10, wherein said tensioning device includes an adjustable self-locking loop and a choking loop.

12. The method of claim 10, wherein said connector includes at least one fixed loop or at least one adjustable self-locking loop.

13. The method of claim 10 wherein a portion of said tensioning device is positioned out of a severed end of said first segment of the severed tendon following (b).

14. The method of claim 10 wherein a portion of said connector is positioned out of a severed end of said second segment of the severed tendon following (d).

15. The method of claim 10, wherein each of said first and said second tissue fixation devices, said tensioning device and said connector is fabricated from a braided material.

16. The method of claim 10, wherein said adjustable self-locking loops of said first and said second tissue fixation devices are spliced loops.

17. The method of claim 16, wherein said spliced loops form whoopie slings.

18. A device for tissue repair comprising a strand formed with an adjustable self-locking loop having two strand tails with a first strand tail forming a choking loop.

19. The device of claim 18, wherein a second strand tail of said adjustable self-locking loop is formed into a fixed loop.
